(19) United States
(12) Patent Application Publication
(10) Pub. No.: US 2016/0250185 A1
(43) Pub. Date: Sep. 1, 2016

(54) PHARMACEUTICAL COMPOSITION FOR CONTROLLING BODY MASS GAIN COMPRISING S-PHENOTROPIL

(71) Applicant: LATVIAN INSTITUTE OF ORGANIC SYNTHESIS, Riga (LV)

(72) Inventors: Ivars KALVINS, Ikskile (LV); Liga ZVEJNIECE, Marupe (LV); Larisa VARACEVA, Riga (LV); Aleksandrs CERNOBROVIJS, Riga (LV); Antons LEBEDEVS, Riga (LV); Maija DAMBROVA, Riga (LV); Baiba SVALBE, Peltes, Sigulda (LV); Edgars LIEPINS, Riga (LV)

(73) Assignee: Latvian Institute of Organic Synthesis, Riga (LV)

(21) Appl. No.: 15/028,794

(22) PCT Filed: Oct. 20, 2014
(86) PCT No.: PCT/LV2014/000011

§ 371 Date: Apr. 12, 2016

(30) Foreign Application Priority Data
Oct. 22, 2013 (LV) P-13-157

(51) Int. Cl. A61K 31/4015; A61K 9/28; A61K 9/48; A61K 9/00

(52) U.S. Cl. CPC A61K 31/4015; A61K 9/0053; A61K 9/28; A61K 9/48

(57) ABSTRACT

The invention relates to medicine, in particular S-Phenotropil ((S)-2-(2-oxo-4-phenylpyrrolidin-1-yl)acetamide) containing pharmaceutical compositions and their use in control of body mass gain.

The invention refers to medicine, in particular to pharmaceutical compositions containing S-Phenotropil ((S)-2-(2-oxo-4-phenylpyrrolidin-1-yl)acetamide)) and the use thereof for the control of body mass gain. One of the unsolved problems of medicine is the prevention and treatment of obesity, especially in patients with diabetes mellitus or metabolic syndrome, as well as in patients, who have an underlying disease that limits their capacity to be involved in physical activities (D. W. Haslam, W. Ph. T. James. Curbing the obesity epidemic, The Lancet, Vol. 367, Issue 9522, 13-19 May 2006, Page 1549).

Attempts to limit the increase in body mass medically by using a range of various pharmacological products, for instance, orlistat, lorcaserin, sibutramine, rimonabant, metformin, exenatide, pramlintide, as well as combinations thereof, like Redux and Phen-phen have been described http://en.wikipedia.org/wiki/Anti-obesity medication.

One of the infrequent medications that are still permitted for use as a body mass control agent is orlistat. It is a drug that blocks the activity of lipases and thus inhibits the absorption of fats from the gastrointestinal tract. Unfortunately the administration of this medicine causes unpleasant sensory experiences in the patient and gastrointestinal disorders, including steatorrhea and it may also cause difficulty in controlling bowel movements. Much more serious adverse effects of orlistat include severe liver damage induced by the medicinal product

(http://www.fda.gov/Drugs/DrugS afety/PostmarketDrugSafetyInformation forPatientsandProviders/ucm213038.htm).

Meanwhile lorcaserin was developed as an anorectic medication, the action of which is connected with its capacity to selectively agonise 5-HT_{2C} receptor (Thomsen, W. J.; Grottick, A. J.; Menzaghi, F.; Reyes-Saldana, H.; Espitia, S.; Yuskin, D.; Whelan, K.; Martin, M. et al. (2008). “Lorcaserin, a Novel Selective Human 5-Hydroxytryptamine2C Agonist: in Vitro and in Vivo Pharmacological Characterisation”. Journal of Pharmacology and Experimental Therapeutics 325 (2): 577-587). Unfortunately this drug causes adverse effects such as headaches, infections of the upper respiratory tract, nasopharyngitis, sinusitis and nausea in patients, while in diabetic patients it may cause hypoglycaemia, back pain, cough and vomiting. This medicinal product has also demonstrated carcinogenicity in experiments with rats and it may cause serotonin syndrome (http://www.fda.gov/NewsEvents/Newsroom/PressAnnouncements/ucm309993.ht m, http://www.sfgate.com/business/article/FDA-staff-says-Arena-diet-pill-linked-to-cancer-3174771.php).

Sibutramine is comparatively widely used in body mass control. It is a CNS-active serotonin-norepinephrine reuptake inhibitor. Unfortunately this medicinal product may cause sudden death of a patient, heart failure, renal infarction and gastrointestinal disorders and therefore it has been withdrawn from the markets of several countries, for instance, the markets of the USA, England, the EU, Australia, Canada and Columbia, (http://en.wikipedia.org/wiki/Anti-obesity_medication).

Marketing of an appetite reducing medicine rimonabant, which is a reversible inhibitor of CB1 cannabinoid receptor, has also been discontinued, because this drug causes serious depression and suicidal tendencies (http://news.bbc.co.uk/2/hi/health/7687311.stm).

Metformin is also used in diabetes patients for weight control. The drug reduces the neosynthesis of glucose in the liver (George A. Bray and Frank L. Greenway (1999). “Current and Potential Drugs for the Treatment of Obesity: Table 19: Clinical trials with metformin for the treatment of obese diabetics”. Endocrine Reviews 20 (6): 805-87). However, this medicine causes severe gastrointestinal disorders, as well as,—like phenformin, it may induce lactic acidosis and health problems associated with this.

Exenatide is a macromolecular substance that is recommended for body mass control. It is a GLP-1 agonist (Glucagon-like peptide-1 agonist), which is usually administered in the therapy of type 2 diabetes mellitus. The active substance of this medication is a synthetic hormone exedin-4 and therefore only parenteral administration of the medicinal product is possible. The hormone amylin and its synthetic analogue pramlintide, which is used for the therapy of diabetes mellitus, demonstrate certain activity in terms of body mass reduction. However, this medication also requires parenteral administration (Jones M C (2007). “Therapies for diabetes: pramlintide and exenatide” American Family Physician 75 (12): 1831-5).

Ranges of serotonin release inducing medicinal products have been used for the control of body mass as well. These include fenfluramine, dexfenfluramine, levofluramine, norfenfluramine, benfluorex, etc. They are amphetamine-like substances without psychostimulatory action. Unfortunately the medicinal products of this type, like sibutramine, have an adverse effect on the heart, especially the heart valves, which are connected with their effect on serotonin 2b receptor. Therefore, the development of an orally administered body mass controlling medicinal product that is suitable for long term use and would not cause addiction or other severe side effect is still an actual issue.

We made an unexpected discovery that the target of the invention may be reached, if pharmaceutical compositions containing S-phenotropil are used for body mass control. A medicinal product R,S-phenotropil (Carphedon) is known in Russia and some of the CIS countries. Racemic R,S-phenotropil is a nootropic medicinal product, which surpasses piracetam 30-60 times in terms of its activity (Malykh A G, Sadaie M R (2010), Piracetam and piracetam-like drugs: from basic science to novel clinical applications to CNS disorders. Drugs 70:287-312). Pharmaceutical compositions that contain Racemic R,S-phenotropil are recommended as medicinal products, which also stimulate the physical capacity of people and tolerance to the influences of cold.

As clinical and experimental studies have demonstrated, phenotropil has other properties characteristic to nootropic medicinal products as well (Malykh A G, Sadaie M R (2010) Piracetam and piracetam-like drugs: from basic science to novel clinical applications to CNS disorders. Drugs 70:287-312). After a long term administration in Wistar rats, phenotropil reduced the manifestations of neuralgic deficit and helped to retain mobility in the research and memory experimental cerebral ischemia model (Tiurenkov I N, Bagmetov M N, Epishina V V (2007) Comparative evaluation of the neuroprotective activity of phenotropil and piracetam in laboratory animals with experimental cerebral ischemia. Eksp Klin Farmakol 70:24-29). Phenotropil also has antiepileptic properties in the metrazol-induced cramps model in mice (Malykh A G, Sadaie M R (2010) Piracetam and piracetam-like drugs: from basic science to novel clinical applications to CNS disorders. Drugs 70:287-312); and it also considerably reduced cramps and EEG changes in epileptic patients, after being used concomitantly with other medicinal products as a complex therapy (Bel'skaia G N, Ponomareva I V, Lukashevich I G, Tikhomirova I N (2007) Complex treatment of epilepsy with phenotropil. Zh Nevrol Psikhiatr Im S S Korsakova 107:40-43; Lybzikova G N, Iaglova Z, Kharlamova I (2008) The efficacy of phenotropil in the complex treatment of epilepsy. Zh Nevrol Psikhiatr Im S S Korsakova 108:69-70).

Although racemic phenotropil can be separated into R- and S-enantiomers, clinically it is still used as a racemic mixture of enantiomers. Levetiracetam, an analogue of the nootropic medicinal product piracetam, is clinically used as an optically clean compound and its anti-cramp effect is manifested in the case of 5-enantiomer (Gower A J, Noyer M, Verloes R, Gobert J, Wulfert E (1992) ucb L059, a novel anti-convulsant drug: pharmacological profile in animals. Eur J Pharmacol 222:193-203). Oxiracetam can also be separated into R- and 5-enantiomers; furthermore, the enantiomers have a stereoselective effect (Almeida J F, Grande M, Moran J R, Anaya J, Mussons M L, Caballero M C (1993) Synthesis of the 3-hydroxy oxiracetam enantiomers, potential nootropic drugs. Tetrahedron: Asymmetry 4:2483-2494). Comparative pharmacological activity in cases of R- and S-phenotropil bears evidence that R-phenotropil has a considerably higher psychostimulating activity than S-phenotropil during experimental locomotor activity and antidepressive effect tests (Zvejniece L, Svalbe B, Veinberg G, Grinberga S, Vorona M, Kalvinsh I, Dambrova M. Investigation into the stereoselective pharmacological activity of phenotropil. Basic Clin Pharmacol Toxicol. 2011;109(5):407-12).

It is also asserted that racemic phenotropil can be used for the control of weight increase in obese patients (http://pharmday.com/index.php?route=product/product&product_id=114).

However, the fact that R,S-phenotropil or carphedon is included into the list of substances prohibited for athletes, since it is admitted to be doping, and that it has a psychostimulant effect, raises concern of the possibility to develop an addiction in the case of long term use of the medicinal product. Therefore, in order to enable the extensive use of such structures for the reduction of body mass and for the control of increase thereof, a medicinal product with the psychostimulant effect reduced to the minimum that also would not affect the motor activity of the patient had to be developed, which is the objective of this invention.

We unexpectedly succeeded in discovering that the objective of the invention can be reached by means of a pharmacological composition, which contains S-phenotropil as an active substance, instead of R,S phenotropil. Although it was known that racemic R,S-phenotropil has the capacity to reduce the increase in body mass, it was not known whether it is possible to create a medicinal product for the control of increase in body mass with a minimised effect on the CNS on the basis of any of the enantiomers of phenotropil, in order to reduce the possible development of addiction and other influences characteristic of the R,S-phenotropil on the functionality of the CNS. The phenomenon that, in the case of most racemic medicinal products that have one or several optically active centres in the site of binding to the receptor, only one of the enantiomers is active- is well known. For instance, in the case of phenibut and baclofen, which are close to phenotropil according to their physiological effect and structure, the CNS activity is manifested by R-enantiomer only, while S-enantiomer is inactive (Dambrova M, Zvejniece L, Liepinsh E, Cirule H, Tharkova O, Veinberg G, Kalvinsh I. Comparative pharmacological activity of optical isomers of phenibut. Eur J Pharmacol. 2008;583(1):128-34).

Upon the tests performed on R,S-phenotropil and the locomotor, antidepressive and cognitive effects of its R- and S-enantiomers, we unexpectedly discovered that phenotropil is an exception, where, contrary to expectations, both enantiomers of phenotropil demonstrated a physiological effect of similar type in most tests, which correlated with the effects of racemic phenotropil as described in the literature. At the same time we unexpectedly discovered that although in a range of tests R- and S phenotropil have similar effects and their concentration in the brain is also similar, considerable differences in the doses triggering such activity exists. We discovered that in an open field test S-phenotropil only exerts a statistically significant effect on locomotor activity in a 10 times higher dose than R-phenotropil. Furthermore S-phenotropil, in contrast to R-phenotropil, does not demonstrate any effect on the function of the CNS during general CNS tests. We also managed to discover that S-phenotropil does not have any effect in a passive avoidance test, while R-phenotropil considerably affects memory. At the same time we succeeded in proving that pharmaceutical compositions containing S-phenotropil have a strongly pronounced effect on the increase in body mass in animals that are genetically predisposed to obesity.

Thus, we unexpectedly discovered that although R- and S-enantiomers of phenotropil have a similar effect on certain physiological processes, S-phenotropil has unexpected and considerable advantages in terms of the possibilities to use pharmaceutical compositions containing this substance for oral control of body mass, because it does not possess the effect on the CNS characteristic to R,S- and R-phenotropil to the extent that could prevent the use of pharmaceutical compositions containing S-phenotropil in medicine for the control of pathologies not directly related to the function of the CNS.

Therefore, we unexpectedly succeeded in proving that in contrast to racemic R,S-phenotropil and R-phenotropil, pharmaceutical compositions containing S-phenotropil ensure the reaching of the goal of the invention development of a highly efficient medication for body mass control without considerable adverse effects on the functions of the CNS, which would also not have a psychostimulating effect characteristic of doping.

The subject matter of the invention is characterised, but not limited by the following examples.

S-phenotropil was used in the experiments. It was obtained by asymmetric synthesis from S-2-ethoxycarbonyl-3-phenyl-4-nitrobutyric acid ethyl ester, which was prepared as follows: 3.2 g of magnesium triflate are suspended in 1,000 ml of chloroform, 0.7 ml of water, 4.2 g of (3aS,3′aS,8aR,8′aR)-2,2′-cyclopropylidenbis-[3a,8a]dihydro-8H-indeno[1,2-d]oxazole, 35.7 g of molecular sieve 4 Å are added and stirred for 30 min at 20° C. Then 29.8 g of β-nitrostyrol, 35.2 g of diethyl malonate and 1.3 g of N-methylmorpholine are added. The obtained solution is stirred at 20° C. until the reaction with β-nitrostyrol is completed, the reaction mixture is filtered, the precipitate is washed with chloroform, the solution is treated with 50 g of silica gel, filtered and the product is separated from the precipitate by using chloroform and evaporated, then hexane is added and the solution is cooled down while stirring intensively. The crystalline mass is filtered, washed with a small amount of cold hexane and air dried at 20° C. 55.7 g of the product are obtained (90%; ee 95-98%). The product is recrystallised from hexane/ethyl acetate mixture and 47.0 g of S-2-ethoxycarbonyl-3-phenyl-4-nitrobutyric acid ethyl ester (76%; ee->99.9%) are obtained. During the next phase 43.0 g of ethoxycarbonyl-3-phenyl-4-nitrobutyric acid ethyl ester are dissolved in propanol-2 and 10.0 g of 50% suspension of Raney nickel in water, which has been previously rinsed with propanol-2, are added. The reaction mass is stirred in an autoclave in hydrogen atmosphere (H₂ pressure 3-4 atm) at 20° C. temperature until the reaction of S-2-ethoxycarbonyl-3-phenyl-4-nitrobutyric acid ethyl ester has completed. The reaction mixture is filtered; the nickel catalyst is washed with propanol-2. The filtrate is evaporated to the volume of 50-60 ml, cooled to −20° C., until the mass crystallises. Cold propanol-2 is added to the product, filtered, washed with propanol-2 and air dried. As a result 21.0 g (65%) of S-3-ethoxycarbonyl-4-phenylpyrrolidone-2 are obtained. During the next phase 29.05 g of S-3-ethoxycarbonyl-4-phenylpyrrolidone-2 with the optical purity of 96-97% are dissolved in DMF (54 ml), water is added (3.5 ml) and the obtained solution is stirred at the temperature of 130-140° C., distilling the solution of ethanol and water off. 3.5 ml of water are added to the solution every 1-2 hours. When the reaction of the source substance is completed, the reaction solution is cooled down to 60° C., evaporated in vacuum to the volume of 40-50 ml and, poured into water, while stirring intensively. The obtained suspension is cooled down to −5° C., filtered, washed with water and vacuum-dried. 16.66 g (83.3%) of S-4-phenylpyrrolidone-2 are obtained, which is recrystallised from toluene and 12.63 g (63.2%) of S-4-phenylpyrrolidone-2 with the optical purity of 99.5% are obtained. During the next phase 10.46 g of 30% potassium hydride suspension in mineral oil are suspended in 30 ml of dioxane and, while intensively stirring, 11.74 g of S-4-phenylpyrrolidone-2 are added to it at the temperature of 20° C. The obtained suspension is stirred for 1 hour at 90° C., cooled down to 20° C. and 13.07 g of ethyl bromoacetate solution in 30 ml of dioxane is added. The obtained suspension is stirred at the temperature of 125° C. for 5-6 h, cooled down to 20° C., then, while stirring intensively, 150 ml of ethyl acetate are added first, followed by 50 ml of saturated NaCl solution in water, and then it is diluted with water. The upper layer is dried with Na₂SO₄ and evaporated in vacuum. The residue is dissolved in 560 ml of methanol, decanted and the obtained S-N-(ethoxycarbonyl) methyl-4-phenylpirrolidone-2 solution in methanol is saturated with dry ammonia at the temperature of 45° C. and stirred for 5 h. When the reaction of the starting material has completed, the reaction mixture is evaporated, the residue is dissolved in ethyl acetate and repeatedly evaporated. The obtained technical product is dissolved in 500 ml CH₂Cl₂, 20 g of silica gel are added, and the product is stirred for 30 min at the temperature of 20° C., filtered and boiled down. The residue is recrystallised from the ethyl acetate with activated charcoal. 10.35 g of S-phenotropil with the optical purity of 99.85% are obtained (66.6% if counted from S-4-phenylpyrrolidone-2). The product is recrystallised from ethyl acetate and a product with the optical purity of >99.9% is obtained. The obtained S-phenotropil was used for the preparation of pharmaceutical compositions and for the tests of its pharmacological efficacy.

### EXAMPLE 1

Locomotor activity was tested in an open field test 30, 60, 120, 180 and 240 min after a single i.p. injection of the racemic phenotropil and R- and S-enantiomers (FIG. 1A,B,C,D). Dose and time dependent increase in locomotor activity was observed during the open field test, which is demonstrated both by the distance run [F(49.360)=7.781, p<0.0001], and the speed of motion [F(49.360)=7.135, p <0.0001]. R-phenotropil in 5, 10 and 50 mg/kg doses induced a statistically significant increase in locomotor activity in comparison with the control animals [F(3.31)=10.37, p<0.0001] (FIG. 1A,C). Furthermore, R-phenotropil in the dose of 50 mg/kg retained a statistically significant increase in locomotor activity for up to 240 min [F(9.80)=24.03, p<0.0001] (FIG. 1B,D).

Racemic phenotropil was less active than R-phenotropil, but the difference in results was not statistically significant for the respective doses. Racemic phenotropil significantly affected locomotor activity in the case of 10 and 50 mg/kg doses and retained the increase in locomotor activity for up to 180 min (FIG. 1A,B,C,D).

S-enantiomer only increased locomotor activity in the case of a 50 mg/kg dose [F(9.75)=13.91, p<0.0001]. Furthermore, the effect of S-enantiomer in the case of a 50 mg/kg dose was statistically significantly weaker than the effect of R-enantiomer during the open field test [F(9.75)=10.79, p<0.0001] (FIG. 1A,B,C,D).

### EXAMPLE 2

The antidepressive effect of the racemic, R- and S-phenotropil was tested in a forced swimming test (by determining the duration of immobility) 30 min after the i.p. injection of the compounds in doses 10, 50 and 100 mg/kg (FIG. 2). All tested compounds demonstrated an antidepressive effect in comparison with the control animals [F(12.116)=25.05, p<0.0001]. R-phenotropil significantly reduced the duration of immobility in the doses of 50 and 100 mg/kg [F(2.27)=52.99, p<0.0001], the dose of 100 mg/kg reduced the duration of immobility 8 times in comparison with the control group. The effect of R-phenotropil was significantly higher in comparison with racemic and S-phenotropil [F(2.27)=39.13, p<0.0001]. Only 100 mg/kg doses of racemic and S-phenotropil statistically significantly reduced the duration of immobility in comparison with the control group.

### EXAMPLE 3

The influence of racemic, R- and S-phenotropil on memory processes was determined by means of a passive avoidance test (PCR), by registering the latent period of the remembering process. Racemic and R-phenotropil in a 1 mg/kg dose significantly increased the latent period in comparison with the control group (FIG. 3). As can be seen in FIG. 3, the injection of R-phenotropil in mice in the doses of 1 and 10 mg/kg increased the latent period by 195% and 185% respectively, in comparison with the control group (p<0.05). S-phenotropil did not affect the latent period. Furthermore, the latent period in the S-phenotropil group in the dose of 1 mg/kg statistically significantly differed from that of the R-phenotropil group (p<0.05).

### EXAMPLE 4

Inhibiting effect on muscle strength and coordination was observed for R-phenotropil in cylinder, traction and rotating rod tests, where the ED₅₀ for R-phenotropil was 258±25, 250±40 and 320±60 mg/kg respectively (Table 1). Racemic and S-phenotropil did not affect muscle strength and coordination up to the dose of 500 mg/kg. None of the compounds affected rectal temperature at the dose of 500 mg/kg (Table 1).

Effect of the substances on, muscle tone,
coordination and body temperature.

| | Phenotropil racemate | R-phenotropil | S-phenotropil |
|---|---|---|---|
| TESTS | ED₅₀ (mg/kg) | ED₅₀ (mg/kg) | ED₅₀ (mg/kg) |
| Cylinder | >500 | 258 ± 25 | >500 |
| Traction | >500 | 250 ± 40 | >500 |
| Rotating rod | >500 | 320 ± 65 | >500 |
| Rectal | >500 | >500 | >500 |
| temperature | | | |

The compounds were administered in i.p. doses of 50, 100, 250 and 500 mg/kg. The effect of the substances in different tests was evaluated 30, 60, 120 and 180 min after the administration of the compounds. ED₅₀ value was calculated by, performing probit analysis.

### EXAMPLE 5

The influence of an S-phenotropil containing composition on the body mass. Zucker fa/fa (HsdOla:Zucker-Lepfre; Harlan Laboratories) and Zucker lean line rats (HsdOla:Zucker-Lean; Harlan Laboratories) with the body mass of 100-170 g were used during the experiment. At the beginning of the experiment the animals were 6 weeks old. Standard conditions for keeping the experimental animals were ensured (air temperature 21° C.-23° C., relative air humidity 65%±10%, 12 hour light/darkness cycle). Standardised feed (R70) by the company LABFOR (Lactamin AB, Sweden) was used to feed the animals, drinking water was accessible without limitations.

S-phenotropil containing pharmaceutical composition in water or water was orally administered to the mature male rats included into the experimental group once daily in the morning: To the Zucker lean group (n=10), water p.o., to the Zucker fa/fa control group (n=10), water p.o. or to the Zucker fa/fa group S-phenotropil containing composition 50 mg/kg (n=5), p.o. The resulting values were expressed as mean values±standard error of the mean (S.E.M.) T-test was used to check the validity of the results. The results were considered valid, if the p value was less than 0.05. As FIG. 1 demonstrates, the administration of S-phenotropil during the 12 weeks of the experiment statistically significantly reduced the weight increase in Zucker FF rats. As FIG. 2 demonstrates, S-phenotropil reduced the total weight gain by 22%. Since the increase in mass for ZuckerLean control rats is primarily connected with normal increase in muscle mass, as the animals grow, the effect of S-phenotropil in terms of the increase of ZuckerFF fat mass is 39% (FIG. 6).

Result table

| | Weight increase in | Increase in fat mass in |
|---|---|---|
| Group | Week 12, AUC units | Week 12, g |
| ZucLean | 1877 ± 69 | 0 |
| ZucFF-K | 3060 ± 60* | 173 ± 10 |
| ZucFF-S-fen | 2645 ± 30*^{, #} | 106 ± 7 ^{#} |

Thus, experiments in vivo convincingly prove that the patentable pharmaceutical composition that contains S-phenotropil allows reaching the objective of the invention—ensuring considerable reduction in the body mass growth in genetically predisposed animals.

Meanwhile pharmaceutical compositions of S-phenotropil for oral administration, either coated or uncoated, in the form of capsules, caplets, tablets, granules, dragees or as solutions, syrups and other orally administered pharmaceutical forms are suitable for use. Traditional methods of manufacturing pharmaceutical forms may be used in the production thereof. Preference shall be given to the compositions that are suitable for the preparation of orally administered pharmaceutical forms, as well as syrups and solutions that contain S-phenotropil and excipients.

For instance, one of the possible pharmaceutical compositions that illustrates this invention is the following composition containing S-phenotropil for the manufacturing of tablets:

| | |
|---|---|
| S-phenotropil | 50 mg |
| Starch | 18 mg |
| Ca-stearate | 1.5 mg |
| Lactose | 80.5 mg |
| Total | 150 mg |

The illustration of the content of the composition suitable for the manufacturing of capsules is as follows:

| | |
|---|---|
| S-phenotropil | 100 mg |
| Lactose | 20 mg |
| Starch | 12 mg |
| Talc | 3 mg |
| Ca-stearate | 5 mg |
| Total | 140 mg |

If S-phenotropil or its pharmaceutically acceptable salt is administered as an injection or as drops, syrup or a drink orally, the pharmaceutical composition shall contain phenotropil in the total amount of 0.01 to 20% by mass and any of the pharmaceutically permissible diluents, for instance—distilled water, isotonic, glucose or buffer solution.

If the active substance is administered in tablets, caplets, dragees, granules, powders or capsules, they shall contain S-phenotropil or its pharmaceutically acceptable salt relative to the total amount from 0.01 to 0.1 g in a tablet, caplet, dragee, and capsule or in one dose of powder or granules.

1) A pharmaceutical composition for controlling body mass gain in humans and animals comprising S-phenotropil ((S)-2-(2-oxo-4-phenylpyrrolidin-1-yl)acetamide) in admixture with a pharmaceutically acceptable adjuvant, diluent or carrier.

2) The pharmaceutical composition according to claim 1, for oral or sublingual administration in a form of tablet, coated tablet, capsule, caplet, dragee or granule, powder or solution that contains 0.01-0.5 g of the active substance by weight in each tablet, capsule, dragee or a dose of granules or powder or it is a 0.01-20% solution or syrup for oral administration.

3) The pharmaceutical composition according to claim 1, for parenteral administration in a form of sterile solution that contains 0.01-0.5 g of the active substance in each dose for parenteral administration together with a pharmaceutically acceptable carrier.

4) The pharmaceutical composition according to claim 1, which is distinguished by 10-500 mg content of S-phenotropil in it.

5) The pharmaceutical composition according to claims 1 and 3 for parenteral administration distinguished in that solution contains 0.01-10 mg/ml S-phenotropil.

6) The pharmaceutical composition according to claim 1 for prevention and treatment of diseases which have a pathogenesis related to obesity.

7) The pharmaceutical composition according to claim 1 for prevention and treatment of diseases where disease is selected as such which is caused by sedentary lifestyle and increased appetite.

8) The pharmaceutical composition according to claim 1 for prevention and treatment of diseases where diseases belongs to the group of diseases that include metabolic syndrome and diabetes mellitus.

9) A pharmaceutical composition comprising S-phenotropil for prevention and treatment of diseases related to the increase in body mass gain in humans and animals after using medications.
